(19) United States
(12) Patent Application Publication
(10) Pub. No.: US 2025/0170281 A1
(43) Pub. Date: May 29, 2025

(54) ANTI-CANCER TREATMENT WITH A RADIOACTIVE PARP INHIBITOR

(71) Applicant: The Chancellor, Masters and Scholars of the University of Oxford, Oxford, Oxfordshire (GB)

(72) Inventors: Bart Cornelissen, Oxford (GB); Veronique Gouverneur, Oxford (GB); Chung Ying Chan, Oxford (GB); Zijun Chen, Oxford (GB); Gianluca Destro, Oxford (GB); Florian Guibbal, Oxford (GB); Thomas Wilson, Oxford (GB)

(21) Appl. No.: 18/836,658

(22) PCT Filed: Feb. 15, 2023
(86) PCT No.: PCT/GB23/50349

§ 371 Date: Aug. 7, 2024

(30) Foreign Application Priority Data
Feb. 15, 2022 (GB) 2202006.9

(51) Int. Cl. A61K 51/04; A61P 35/00

(52) U.S. Cl. CPC A61K 51/0459; A61P 35/00

(57) ABSTRACT

The present invention relates to a novel anti-cancer radiopharmaceutical treatment that is particularly suitable for targeting PARP enzyme expressing cancers. The novel treatment involves PARP-targeted Auger-emitting radiopharmaceutical compounds of formula (I), or a pharmaceutically acceptable salt or solvate thereof:

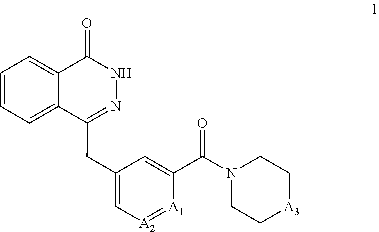

wherein:
A₁, A₂ and A₃ are each as defined herein. The present invention also relates to processes for the preparation of these compounds, to pharmaceutical compositions comprising them, and to their use in the treatment cancer.

### INTRODUCTION

The present invention relates to a novel anti-cancer radiopharmaceutical treatment. The anticancer treatment is particularly suitable for targeting PARP enzyme expressing cancers. The novel treatment involves PARP-targeted Auger-emitting radiopharmaceutical compounds and the present invention relates to these compounds, processes for making these compounds, their therapeutic uses and/or their use as imaging agents.

### BACKGROUND OF THE INVENTION

Cancer is caused by altered cellular proliferation, and is characterised by increased genomic instability. The expression of the DNA damage repair enzyme PARP and closely related enzymes is increased in tumour tissue compared to surrounding normal tissue. PARP enzymes bind to DNA with a single strand break, use the substrate NAD+ and catalytically convert the latter to adenosyl ribose adducts on target proteins. Compounds binding PARP enzymes and hinder binding of NAD+ can inhibit PARP and trap the enzyme to the DNA.

There remains a need for new and effective treatments for cancer. In particular, there is a need for new and effective treatments for hard-to-treat cancers such as pancreatic ductal adenocarcinoma and glioblastoma, a significant proportion of which are PARP enzyme-expressing cancers.

The present invention was devised with the foregoing in mind.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a cancer in which PARP enzyme expression is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to a further aspect of the present invention, there is provided a compound, or a pharmaceutically acceptable salt, hydrate or solvate thereof, or a pharmaceutical composition as defined herein for use in the treatment of cancer.

According to a further aspect of the present invention, there is provided a compound, or a pharmaceutically acceptable salt, hydrate or solvate thereof, or a pharmaceutical composition as defined herein for use in the treatment of cancer in which PARP enzyme expression is implicated.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided the use of a compound, or a pharmaceutically acceptable salt, hydrate or solvate thereof, as defined herein in the manufacture of a medicament for the treatment of cancer in which PARP enzyme expression is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

References herein to “PARP” or “PARP enzyme” refer to all isoforms of the PARP enzyme, including isoforms PARP1 through to PARP17.

A “therapeutically effective amount” means the amount of a compound that, when administered to a mammal for treating a disease, is sufficient to affect such treatment for the disease. The “therapeutically effective amount” will vary depending on the compound, it's Molar Activity, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus. “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include-CH₂F, —CHF₂ and —CF₃.

The term “halo” or “halogeno” refers to fluoro, chloro, bromo, iodo and astatine, suitably fluoro, chloro, bromo and iodo, more suitably, fluoro, chloro and iodo.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl (m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to compounds, or pharmaceutically acceptable salts, hydrates or solvates thereof, having the structural formula (I), shown below:

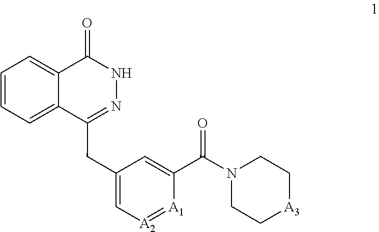

wherein:
A₁ and A₂ are selected from CR₁ and CH, CR₁ and CF, CR₁ and N, CH and CR₁, CF and CR₁ or NH and CR₁ respectively;
R₁ is a radioisotope of iodine (I) or astatine (At);
A₃ is selected from N—X₁—R₃ or C(R₄)OR₅;
X₁ is absent or selected from —C(O)—, —[CH₂]₁₋₃—, —C(O)NH—, —C(O)O—, or —S(O)₀₋₂—;
R₃ is H, (1-10C)alkyl, (3-8C)cycloalkyl, aryl, heterocyclyl or heteroaryl, wherein each alkyl, cycloalkyl, aryl, heterocyclyl or heteroaryl group is optionally substituted by one or more R_{A} substituents;
R₄ is selected from H, or C₁₋₄ alkyl; and
R₅ is selected from (1-7C)alkyl, heterocyclyl and aryl, which groups are optionally substituted by one or more R_{A} substituents;
or R₄ and R₅ together with the carbon and oxygen atoms to which they are attached form a spiro-C₅₋₇ oxygen-containing heterocyclyl, which is optionally substituted by one or more R_{A} substituents or fused to a phenyl or 5 or 6 membered heteroaryl ring; and
each R_{A} substituent is independently selected from fluoro, cyano, (1-4C)alkyl, (1-4C)haloalkyl, (1-4C)hydroxyalkyl, phenyl, 4 to 6 membered heterocyclyl, 5 to 6-membered heteroaryl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), —SO₂N(R_{1b})R₁ₐ, —N(R_{1b})SO₂R₁ₐ wherein R₁ₐ and R_{1b} are each independently selected from hydrogen, (1-4C)alkyl or (3-6C)alkyl.

Particular compounds of the invention include, for example, compounds of the Formula (I), or pharmaceutically acceptable salts, hydrates and/or solvates thereof, defined herein having one of the structural formulae Ia to Ij shown below:

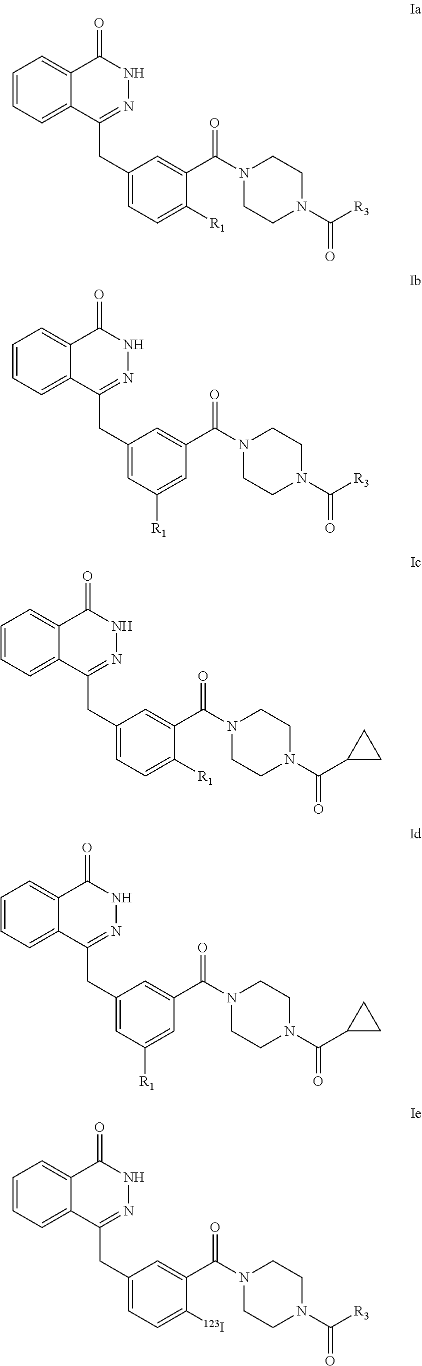

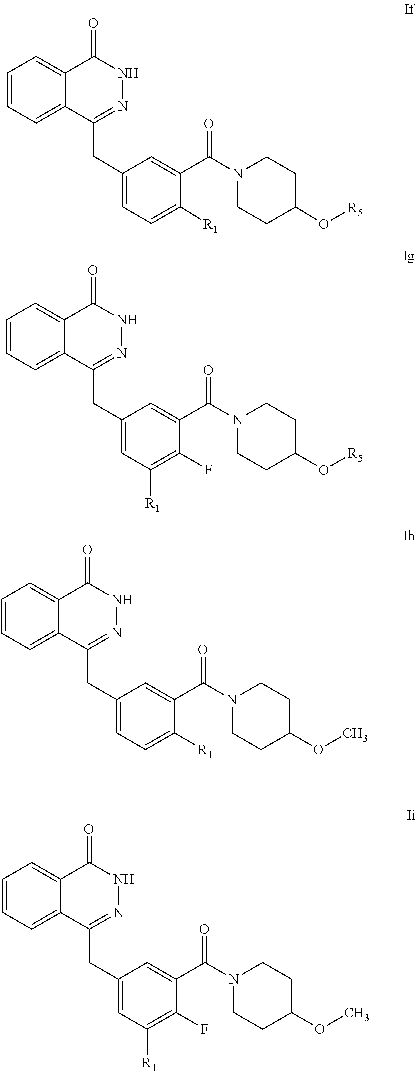

wherein, as appropriate, R₁, R₃ and R₅ are each as defined herein.

Particular compounds of the invention include, for example, compounds of the Formula (I) [including sub-formulae Ia to Ii], or pharmaceutically acceptable salts, hydrates and/or solvates thereof, wherein, unless otherwise stated, each of A₁, A₂, A₃, R₁, X₁, R₃, R₄, R₅ and R_{A} has any of the meanings defined hereinbefore or in any of paragraphs (1) to (34) hereinafter:
(1) A₁ and A₂ are selected from CR₁ and CH, CR₁ and CF, CH and CR₁ or CF and CR₁ respectively;
(2) A₁ and A₂ are selected from CR₁ and CH, CR₁ and N, CH and CR₁ or NH and CR₁ respectively;
(3) A₁ and A₂ are selected from CR₁ and CH, CH and CR₁ or CF and CR₁ respectively;
(4) A₁ is CR₁ and A₂ is CH;
(5) R₁ is a radioisotope of iodine (I);
(6) R₁ is a radioisotope of astatine (At);
(7) R₁ is selected from ¹²³I, ¹³¹I, ¹²⁷I, ¹²⁴I or ²¹¹At, or ¹²³I, ¹³¹I, ¹²⁴I or ²¹¹At
(8) R₁ is selected from ¹²³I, ¹³¹I or ²¹¹At;
(9) R₁ is ¹²³I or ¹³¹I
(10) R₁ is ¹²³I;
(11) A₃ is N—X₁—R₃;
(12) A₃ is C(R₄)OR₅;
(13) X₁ is absent or selected from —C(O)—, —[CH₂]₁₋₃- or —C(O)NH—;
(14) X₁ is-C(O)—;
(15) R₃ is H, (1-6C)alkyl, (3-8C)cycloalkyl, phenyl, 4 to 10 membered heterocyclyl or 5 or 6 membered heteroaryl, wherein each alkyl, cycloalkyl, aryl, heterocyclyl or heteroaryl group is optionally substituted by one or more R_{A} substituents;
(16) R₅ is H, (1-6C)alkyl, (3-8C)cycloalkyl, phenyl, 4 to 8 membered heterocyclyl or 5 or 6 membered heteroaryl, wherein each alkyl, cycloalkyl, aryl, heterocyclyl or heteroaryl group is optionally substituted by one or more R_{A} substituents;
(17) R₃ is (1-6C)alkyl or (3-8C)cycloalkyl, each of which is optionally substituted by one or more R_{A} substituents;
(18) R₃ is (3-8C)cycloalkyl which is optionally substituted by one or more R_{A} substituents;
(19) R₃ is (3-6C)cycloalkyl which is optionally substituted by one or more R_{A} substituents;
(20) R₃ is cyclopropyl;
(21) R₄ is selected from H, or C₁₋₄ alkyl;
(22) R₄ is selected from H or methyl;
(23) R₄ is H;
(24) R₅ is selected from (1-7C)alkyl, (3-8C)cycloalkyl, 4 to 10 membered heterocyclyl and phenyl, which groups are optionally substituted by one or more R_{A} substituents;
(25) R₅ is selected from (1-7C)alkyl, 4 to 10 membered heterocyclyl and phenyl, which groups are optionally substituted by one or more R_{A} substituents;
(26) R₅ is selected from (1-7C)alkyl, (3-6C)cycloalkyl, 4 to 10 membered heterocyclyl and phenyl, which groups are optionally substituted by one or more R_{A} substituents;
(27) R₅ is (1-6C)alkyl which is optionally substituted by one or more R_{A} substituents;
(28) R₅ is methyl;
(29) R₄ and R₅ together with the carbon and oxygen atoms to which they are attached form a spiro-C₅₋₆oxygen-containing heterocyclyl, which is optionally substituted by one or more R_{A} substituents or fused to a phenyl or 5 or 6 membered heteroaryl ring;
(30) R₄ and R₅ together with the carbon and oxygen atoms to which they are attached form a spiro-C₅₋₆oxygen-containing heterocyclyl;
(31) each R_{A} substituent is independently selected from fluoro, cyano, (1-4C)alkyl, (1-4C)haloalkyl, (1-4C)hydroxyalkyl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), —SO₂N(R_{1b})R₁ₐ, —N(R_{1b})SO₂R₁ₐ wherein R₁ₐ and R_{1b} are each independently selected from hydrogen or (1-4C)alkyl;
(32) each R_{A} substituent is independently selected from fluoro, cyano, (1-4C)alkyl, (1-4C)haloalkyl, (1-4C)hydroxyalkyl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), wherein R₁ₐ and R_{1b} are each independently selected from hydrogen or (1-2C)alkyl;
(33) each R_{A} substituent is independently selected from fluoro, cyano, (1-4C)alkyl, (1-4C)haloalkyl, (1-4C)hydroxyalkyl, or —OR₁ₐ, wherein R₁ₐ is selected from hydrogen or (1-2C)alkyl;
(34) each R_{A} substituent is fluoro.

Suitably, A₁ and A₂ are as defined in any one of numbered paragraphs (1) to (4) above. More suitably, A₁ and A₂ are as defined in numbered paragraphs (3) or (4) above. Most suitably, A₁ and A₂ are as defined in paragraph (4) above.

Suitably, R₁ is as defined in any one of numbered paragraphs (5) to (10) above. More suitably, R₁ is as defined in any one of numbered paragraphs (7) to (10) above. Most suitably, R₁ is as defined in paragraph (10) above.

Suitably, A₃ is as defined in paragraph (11) above.

Suitably, X₁ is as defined in paragraph (13) or (14) above. Most suitably, X₁ is as defined in paragraph (14) above.

Suitably, R₃ is as defined in any one of numbered paragraphs (15) to (20) above. More suitably, R₃ is as defined in any one of numbered paragraphs (18) to (20) above. Most suitably, R₃ is as defined in numbered paragraph (20) above.

Suitably, R₄ is as defined in any one of numbered paragraphs (21) to (23) above. More suitably, R₄ is as defined in numbered paragraphs (22) or (23). Most suitably, R₄ is as defined in numbered paragraph (23) above.

Suitably, R₅ is as defined in any one of numbered paragraphs (24) to (28) above. More suitably, R₅ is as defined in any one of numbered paragraphs (26) to (28). Most suitably, R₅ is as defined in numbered paragraph (28) above.

Suitably, if R₄ and R₅ are both linked, then they are as defined in paragraph (29) or (30) above.

Suitably, R_{A} is as defined in any one of numbered paragraphs (31) to (34) above. More suitably, R_{A} is as defined in numbered paragraphs (33) or (34) above. Most suitably, R_{A} is as defined in paragraph (34) above.

Suitably, R₅ is as defined in any one of numbered paragraphs (28) to (30) above. More suitably, R₅ is as defined in numbered paragraphs (29) or (30) above. Most suitably, R₅ is as defined in numbered paragraph (30) above.

Suitably, y is as defined in numbered paragraph (36) above.

Suitably, x is as defined in numbered paragraph (40) above.

As indicated above, particular compounds of the invention include, for example, compounds of the Formula (I), or pharmaceutically acceptable salts, hydrates and/or solvates thereof, defined wherein having one of the structural formulae Ia to Ij shown above, wherein, as appropriate, R₁, R₃ and R₅ each have any one of the definitions set out herein.

Particular compounds of the invention have structural formulae Ij and Ik shown below:

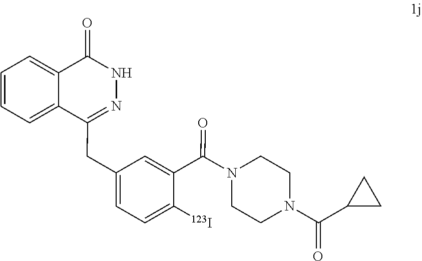 [SMILES: O=C(c1cc(Cc2n[nH]c(=O)c3ccccc23)ccc1[123I])N1CCN(C(=O)C2CC2)CC1]

(also referred to herein as CC1 (4-[(3-[(4-cyclopropylcarbonyl)piperazin-1-yl]carbonyl-4-iodophenyl]methyl(2H)phthalazin-1-one)

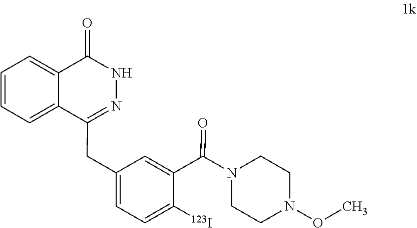 [SMILES: CON1CCN(C(=O)c2cc(Cc3n[nH]c(=O)c4ccccc34)ccc2[123I])CC1]

or pharmaceutically acceptable salts, hydrates and/or solvates thereof.

A particular compound of the invention has the structural formulae Ij above, or pharmaceutically acceptable salts, hydrates and/or solvates thereof.

The various functional groups and substituents making up the compounds of the Formula (I), or sub-formulae (Ia) to (Ie), are typically chosen such that the molecular weight of the compound of the formula (I) does not exceed 1000.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers and mixtures thereof that possess anti-cancer activity or are suitable as imaging agents.

The present invention also encompasses compounds of the invention as defined herein which comprise one or more isotopic substitutions. For example, H may be in any isotopic form, including ¹H, ²H(D), and ³H(T); C may be in any isotopic form, including ¹²C, 13C, and ¹⁴C; and O may be in any isotopic form, including ¹⁶O and ¹⁸O; and the like.

It is also to be understood that certain compounds of the Formula (I), or sub-formulae Ia to Ik, may exist in solvated as well as unsolvated forms such as, for example, hydrated forms. It is to be understood that the invention encompasses all such solvated forms that possess activity.

It is also to be understood that certain compounds of the Formula (I), or sub-formulae Ia to Ik, may exhibit polymorphism, and that the invention encompasses all such forms that possess activity.

Compounds of the Formula (I), or sub-formulae Ia to Ik, may exist in a number of different tautomeric forms and references to compounds of the Formula (I), or sub-formulae Ia to Ik, include all such forms. For the avoidance of doubt, where a compound can exist in one of several tautomeric forms, and only one is specifically described or shown, all others are nevertheless embraced by Formula (I), or sub-formulae (Ia) to (Ie). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

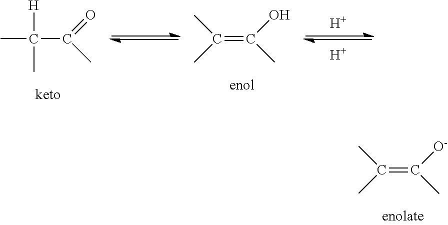

Compounds of the Formula (I), or sub-formulae Ia to Ik, containing an amine function may also form N-oxides. A reference herein to a compound of the Formula (I), or sub-formulae Ia to Ik, that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae Ia to Ik, may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae Ia to Ik, and in-vivo cleavable amide derivatives that may be formed at a carboxy group or an amino group in a compound of the Formula (I), or sub-formulae Ia to Ik.

Accordingly, the present invention includes those compounds of the Formula (I), or sub-formulae Ia to Ik, as defined hereinbefore, when made available by organic synthesis and when made available within the human or animal body by way of cleavage of a pro-drug thereof. Accordingly, the present invention includes those compounds of the Formula (I), or sub-formulae Ia to Ik, that are produced by organic synthetic means and also such compounds that are produced in the human or animal body by way of metabolism of a precursor compound, that is a compound of the Formula (I), or sub-formulae Ia to Ik, may be a synthetically-produced compound or a metabolically-produced compound.

A suitable pharmaceutically acceptable pro-drug of a compound of the Formula (I), or sub-formulae Ia to Ik, is one that is based on reasonable medical judgement as being suitable for administration to the human or animal body without undesirable pharmacological activities and without undue toxicity.

Various forms of pro-drug have been described, for example in the following documents: —
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae Ia to Ik, that possesses a carboxy group is, for example, an in vivo cleavable ester thereof. An in vivo cleavable ester of a compound of the Formula (I), or sub-formulae Ia to Ik, containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae Ia to Ik, that possesses a hydroxy group is, for example, an in vivo cleavable ester or ether thereof. An in vivo cleavable ester or ether of a compound of the Formula (I), or sub-formulae Ia to Ik, containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl. N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I), or sub-formulae Ia to Ik, that possesses a carboxy group is, for example, an in vivo cleavable amide thereof, for example an amide formed with an amine such as ammonia, a (1-4C)alkylamine such as methylamine, a [(1-4C)alkyl]₂ₐ mine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I), or sub-formulae Ia to Ik, that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae Ia to Ik, may be exerted in part by one or more metabolites that are formed within the human or animal body after administration of a compound of the Formula (I), or sub-formulae Ia to Ik. As stated hereinbefore, the in vivo effects of a compound of the Formula (I), or sub-formulae Ia to Ik, may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae Ia to Ik, will vary depending on the nature of the variable groups A₁, A₂ or A₃.

In a particular process of the invention, the process of synthesising a compound of formula I comprises reacting a compound of formula A or B shown below:

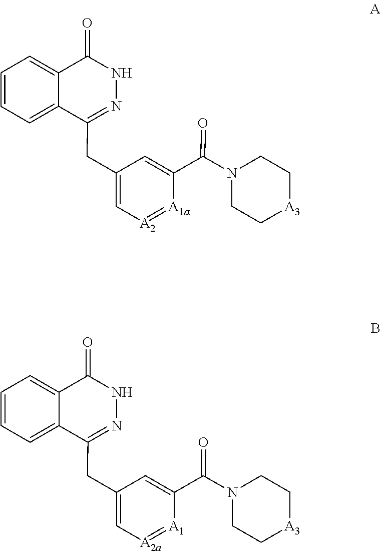

wherein A₁ₐ or A₂ₐ is a group C—B, wherein B is a boronic acid or a boronate ester (e.g. boronate pinacol ester); and A₁ and A₂ are each as defined herein;
with a compound of the formula:

M[R₁]
wherein M is a metal (e.g. Na) and R₁ is as defined herein before;
in the presence of a copper catalyst (e.g. Cu(OCOCF₃)) and a suitable ligand (e.g. 1,10 phenanthroline).

In an embodiment, the reaction involves reacting a compound of formula A in the process defined above.

Suitably, B is a boronate ester (e.g. boronate pinacol ester).

Suitably, M is Na, the copper catalyst is Cu(OCOCF₃) and the ligand is 1,10 phenanthroline.

Suitably, R₁ is ¹²³I.

Suitably, the reaction is conducted in a suitable solvent, e.g. a mixture of methanol and water.

Suitably, the reaction is conducted at an elevated temperature of 50 to 85° C. (e.g. 80° C.) for 10 to 120 minutes (e.g. for 25 minutes).

The compound of the formula Ij defined herein is particularly challenging to synthesise. In a particular process of the invention, the process of synthesising a compound of formula Ij, or a pharmaceutically acceptable salt thereof, comprises reacting a compound of formula Aj shown below:

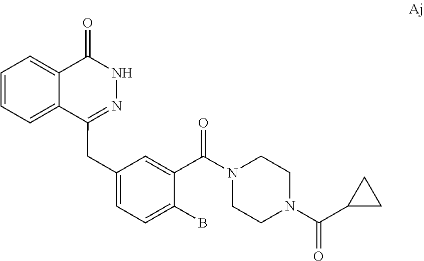

wherein B is a boronic acid or a boronate ester (e.g. boronate pinacol ester); and A₁ and A₂ are each as defined herein;
with a compound of the formula:

M[¹²³I]
wherein M is a metal (e.g. Na);
in the presence of a copper catalyst (e.g. Cu(OCOCF₃)) and a suitable ligand (e.g. 1,10 phenanthroline).

Suitably, B is a boronate ester (e.g. boronate pinacol ester).

Suitably, M is Na, the copper catalyst is Cu(OCOCF₃) and the ligand is 1,10 phenanthroline.

Suitably, the reaction is conducted in a suitable solvent, e.g. a mixture of methanol and water.

Suitably, the reaction is conducted at an elevated temperature of 50 to 85° C. (e.g. 80° C.) for 10 to 120 minutes (e.g. for 25 minutes).

Once a compound of Formula (I), or sub-formulae Ia to Ik, has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups may be further reacted to change the nature of the group and provide an alternative compound of Formula (I).

The resultant compounds of Formula (I), or sub-formulae Ia to Ik, can be isolated and purified using techniques well known in the art.

The compounds of Formula (I), or sub-formulae Ia to Ik, may be synthesised by the synthetic routes shown in the Examples section below.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration.

### Uses

Compounds of the invention in which R₁ is selected from ¹²³I, ¹²⁴I, ¹³¹I, ¹²⁷I, or ²¹¹At are suitable for the therapeutic uses and applications set out below. In particular, compounds of the invention in which R₁ is ¹²³I are exceptionally potent and effective therapeutic agents. In particular, the compound of the formula Ij is demonstrated to be exceptionally active in the examples set out herein.

Compounds of the invention in which R₁ is ¹²⁴I are suitable for PET imaging applications. PET imaging is well-known in the art and compounds of the invention in which R₁ is ¹²⁴I are suitable agents for the PET imaging of cancers, particularly PARP enzyme expressing cancers.

Such compounds can be administered to the body and accumulate in the cancer. The compositions for administering such compounds are the same as the pharmaceutical compositions defined herein. Similarly, the route of delivery such compounds are the same as for the therapeutic compounds defined herein.

Compounds in which R₁ is ¹²³I described herein could be used for therapeutic applications (as noted above) and imaging applications. For example, such compounds could be used, for example, for dosimetry studies, patient selection and/or treatment follow-up.

### Therapeutic Uses and Applications

The present invention provides compounds that are radiopharmaceuticals and are capable of delivering a lethal dose of radiation to tumour cells. Suitably, the compounds of the invention suitable for therapeutic applications are those in which R₁ is ¹²³I, ¹³¹I or ²¹¹At, and especially ¹²³I.

As a consequence, the compounds of the present invention are suitable for treating cancer.

The compounds of the present invention are particularly suited to targeting PARP enzyme expressing cancers.

According to an aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to an aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound or a pharmaceutically acceptable salt, hydrate or solvate thereof as defined herein, or a pharmaceutical composition as defined herein, for use in the treatment of cancer.

According to a further aspect of the present invention, there is provided the use of a compound, or a pharmaceutically acceptable salt, hydrate or solvate thereof, as defined herein in the manufacture of a medicament for the treatment of cancer.

According to a particular aspect of the present invention, there is provided a method of treating a PARP enzyme expressing cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further particular aspect of the present invention, there is provided a compound or a pharmaceutically acceptable salt, hydrate or solvate thereof as defined herein, or a pharmaceutical composition as defined herein, for use in the treatment of a PARP enzyme expressing cancer.

According to a further particular aspect of the present invention, there is provided the use of a compound, or a pharmaceutically acceptable salt, hydrate or solvate thereof, as defined herein in the manufacture of a medicament for the treatment of a PARP enzyme expressing cancer.

The compounds of the present invention may be used to treat any suitable cancer that is susceptible to treatment. Examples of cancers that may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

The compounds of the present invention are particularly suited to the treatment of PARP enzyme expressing cancers. Particular examples of PARP enzyme expressing cancers include glioma, glioblastoma, thyroid cancer, lung cancer (e.g. NSCLC), oesophageal cancer, head and neck cancer, tongue cancer, stomach cancer, liver cancer (e.g. HCC), neuroendocrine cancer, pancreatic cancer (e.g. PDAC), colon cancer, renal cancer, prostate cancer, breast cancers (e.g. TNBC, ductal and invasive subtypes), endometrial cancer, cervical cancer, ovarian cancer, melanoma/skin cancer and lymphoma.

### Dosage

The compounds of the present invention suitable for therapeutic use may be administered at any suitable dose. For example, the compound of the invention may be administered at a dosage of 0.1 MBq to 10 GBq per cycle over 1 to 10 separate treatment cycles. A treatment cycle typically comprises a fixed period of treatment (of, for example 1 to 7 days), followed by a colling off period (of, for example, 1 to 21 days) where no treatment is administered.

In a particular embodiment, the compound of the formula Ij is administered at a dosage of 0.1 MBq to 10 GBq per cycle over 1 to 10 treatment cycles.

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery, additional conventional radiotherapy or chemotherapy. Such chemotherapy may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy) quinazolin-4-amine (gefitinib, ZD1839), N-(3-ethynylphenyl)-6,7-bis(2-methoxyethoxy) quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R₁₁₅₇₇₇) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034).
(vi) vascular damaging agents such as, but not limited to, Combretastatin A₄ and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, A₂ₐ receptor antagonists, PI3 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and a further anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

### EXAMPLES—PART A

### Figure Legends

FIG. 1: (A) Chemical structure of the PARP inhibitors olaparib and CC1. (B) Enzymatic inhibition of PARP1, 2 and 3 of CC1 and a selection of known PARP inhibitors. (C) Heatmap visualizing enzymatic activity screening of CC1 (10 mM) against 13 related enzymes. (D) Radiosynthesis and structure of [¹²³I]CC1. (E) Uptake of [¹²³I]CC1 in AsPC1, PSN1 and U87MG cells. (F) Blocking of [¹²³I]CC1 uptake in AsPC1, PSN1 and U87MG cells by a panel of cold, unlabelled PARP, ALDH2 or IMPDH2 inhibitors. Asterisks indicate levels of significance: ns, P>0.05: *, P<0.05; **, P<0.01; ***, P<0.001; and ****, P<0.0001.

FIG. 2: (A, B) Flow cytometry measurements of PARP1, 2 and 3, and γ-H2AX expression, 0, 1 or 24 h after a 1-hour exposure to [¹²³I]CC1 (50 kBq, 20 GBq/μmol) in AsPC1, PSN1 and U87MG. (C) Clonogenic assays comparing exposure of cell for 1 h to [¹²³I]CC1, or an equivalent concentration of CC1 or olaparib.

FIG. 3: (A) Representative dynamic SPECT images after intravenous administration of [¹²³I]CC1 (3 MBq, 20 GBq/μmol). The middle of time frames is indicated in minutes. Images are presented as maximum intensity projections. (B) VOI analysis of dynamic imaging. (C) Biodistribution of [¹²³I]CC1 in selected tissues in PSN1 xenografts bearing-mice, 1, 2 or 24 h after intravenous administration of [¹²³I]CC1 (3 MBq, 20 GBq/μmol).

FIG. 4: (A) Biodistribution in selected tissues in PSN1 xenograft-bearing mice 2 h after intravenous injection of [¹²³I]CC1 (3 MBq, Aₘ=20 GBq/μmol) (n=3/group), with or without an excess of cold, unlabelled rucaparib (0.5 mg). (B) Autoradiography of PSN1 tumour sections showing ¹²³I localisation. Asterisks indicate levels of significance: ns, P>0.05; *, P<0.05; **, P<0.01; ***, P<0.001; and ****, P<0.0001.

FIG. 5: (A) Tumour growth following a single administration of CC1 or [¹²³I]CC1 (3 MBq, 20 GBq/μmol). (B) Kaplan-Meier survival plots following administration of CC1 or [¹²³I]CC1. (C) Animal weights following administration of CC1 or [¹²³I]CC1.

### SUPPLEMENTARY FIGURES

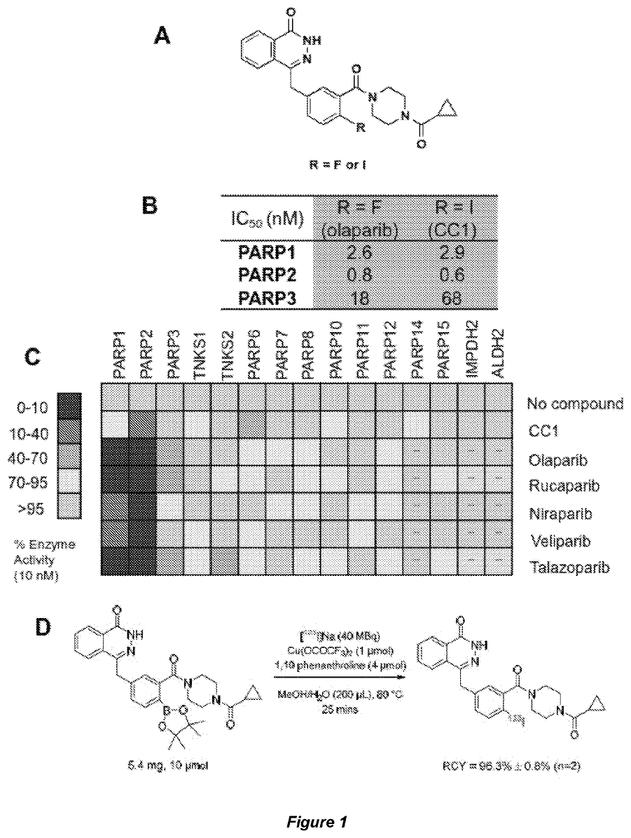

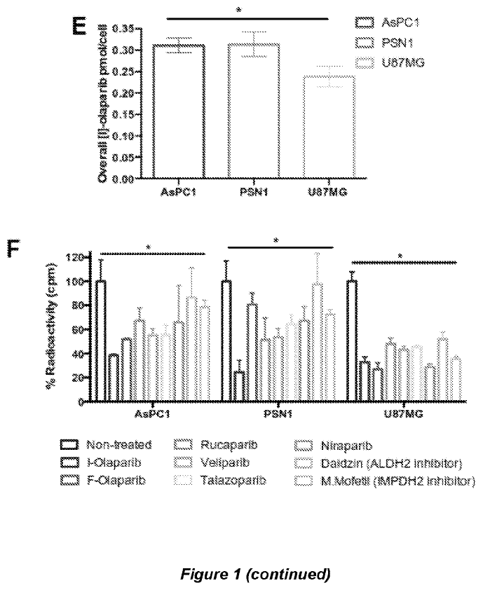

Supplementary FIG. 1: Catalytic inhibition of PARP1, 2, or 3 by increasing concentrations of CC1, olaparib, or rucaparib

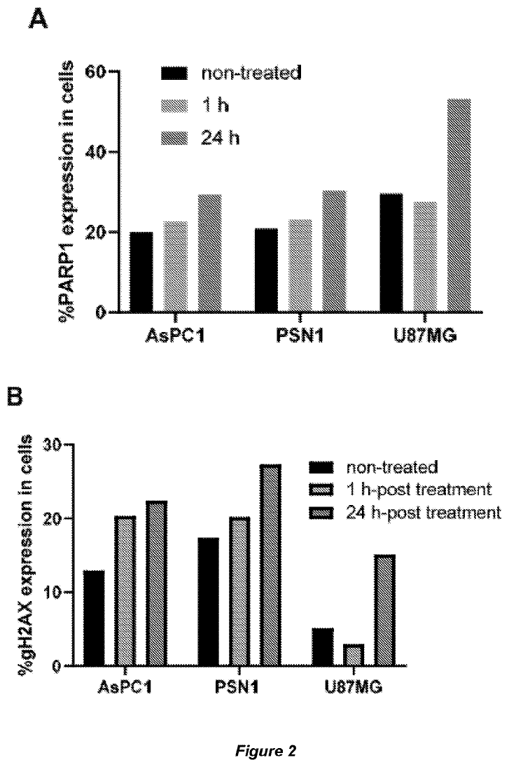

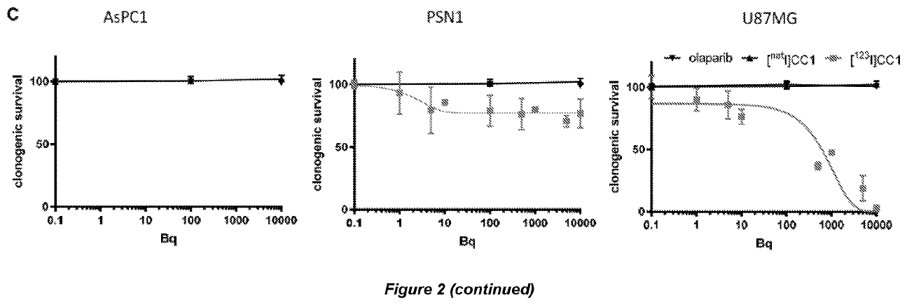

Supplementary FIG. 2: Flow cytometry frequency histogram probing for PARP1, 2 and 3, and IMPDH2 expressions in AsPC1, PSN1 and U87MG cells.

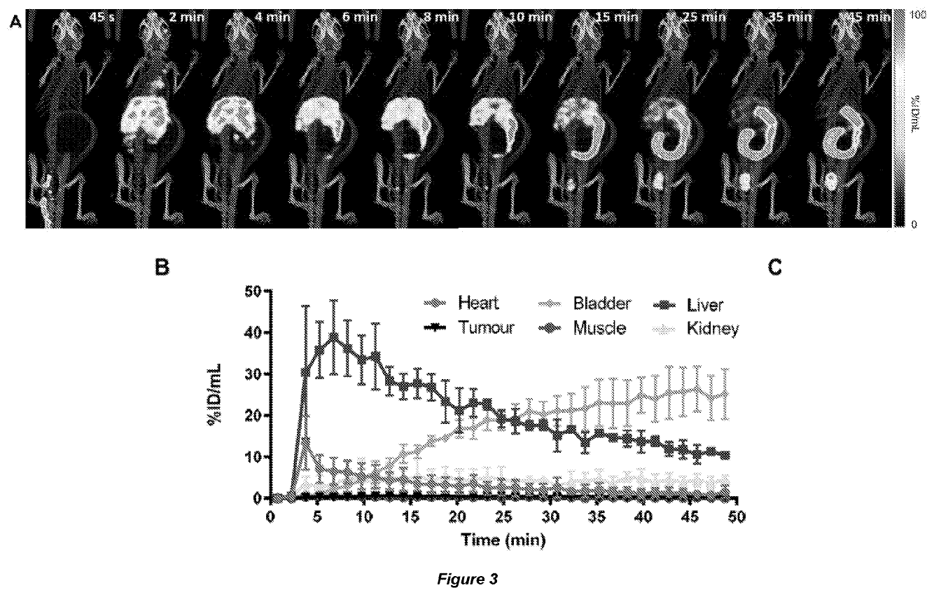

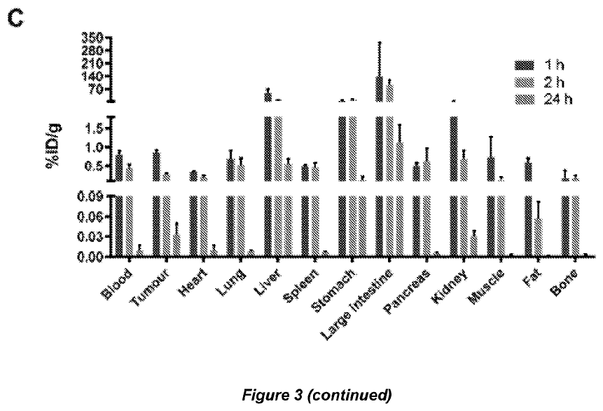

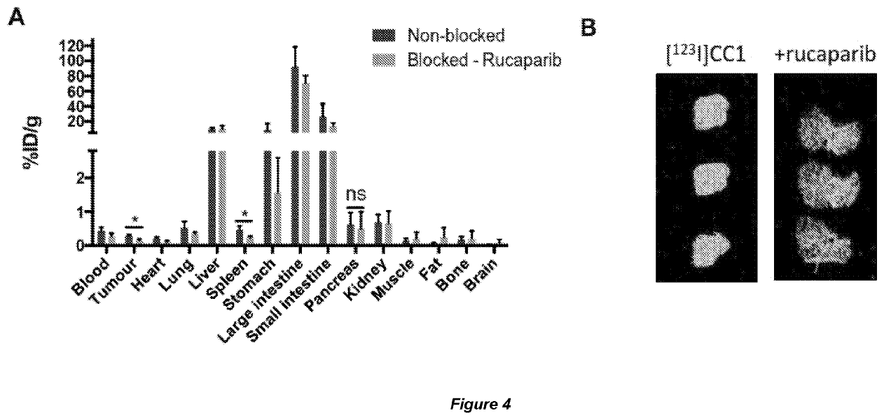

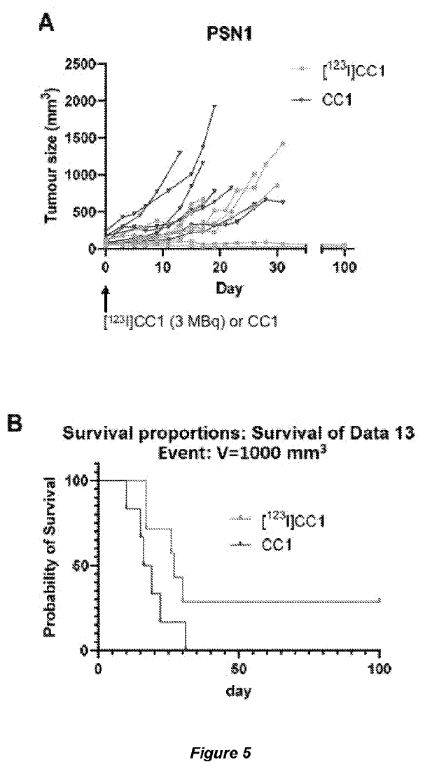

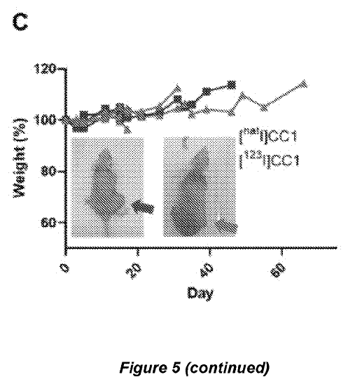

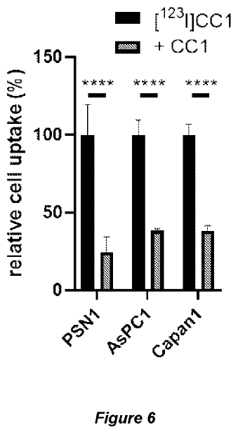

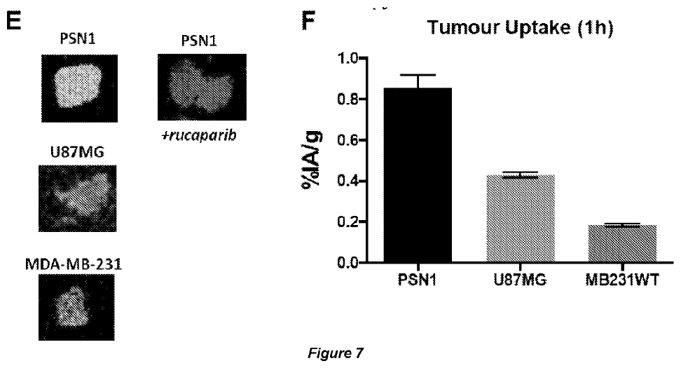

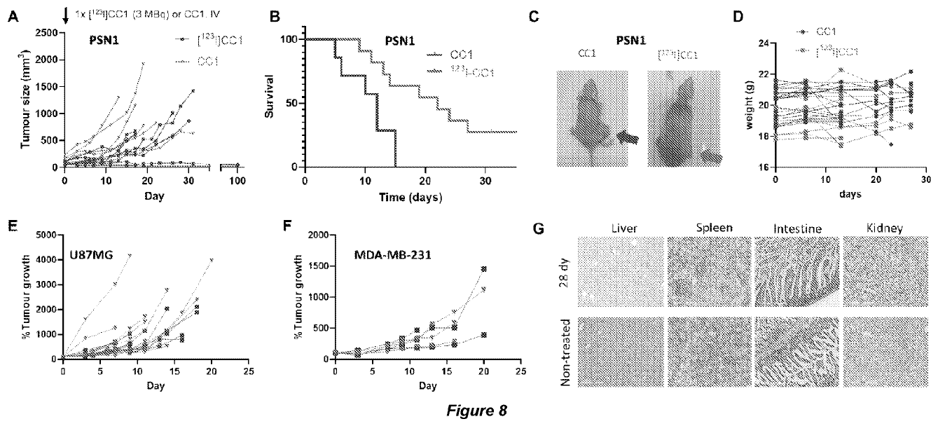

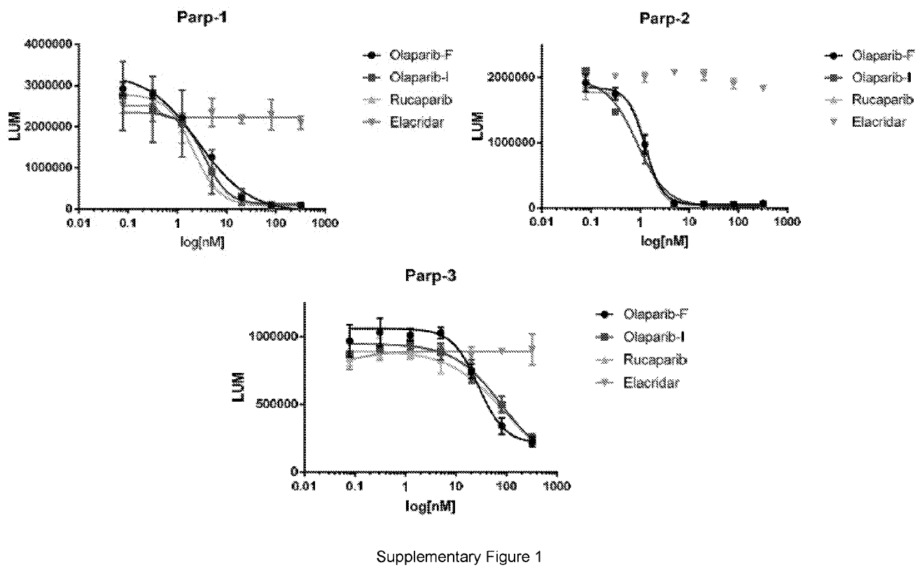

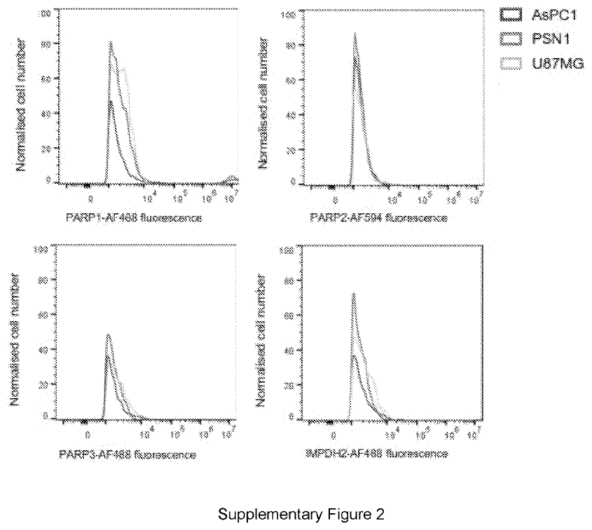

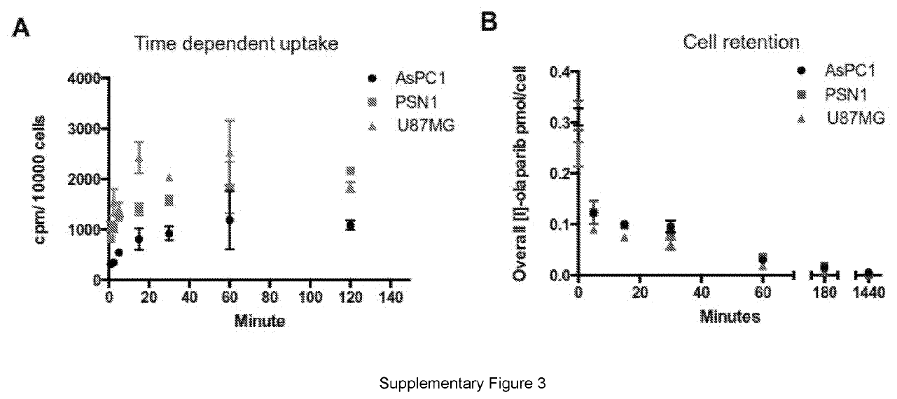

Supplementary FIG. 3: (A) Time dependent uptake of [¹²³I]CC1 in AsPC1, PSN1 and U87MG cells. (B) Cell retention assay of [¹²³I]CC1 in AsPC1, PSN1 and U87MG cells.

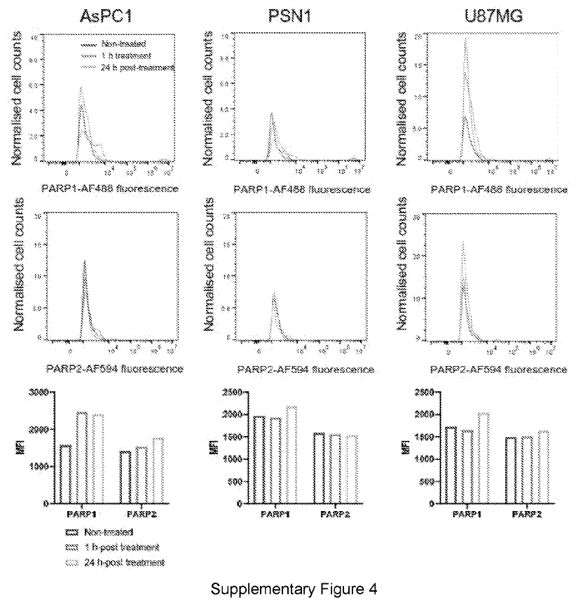

Supplementary FIG. 4: Flow cytometry measuring PARP1, 2, and 3 expression in cells after exposure to [¹²³I]CC1.

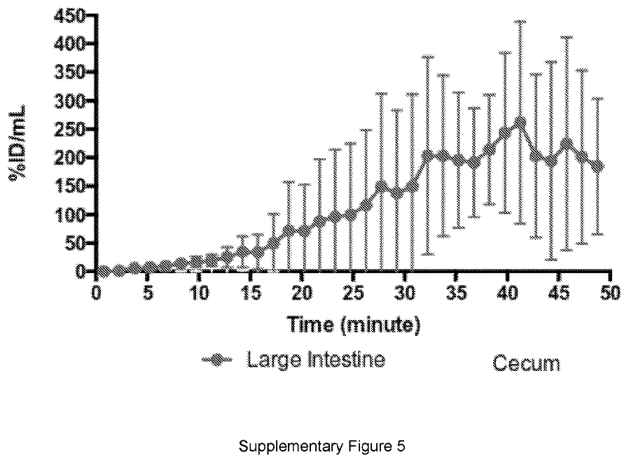

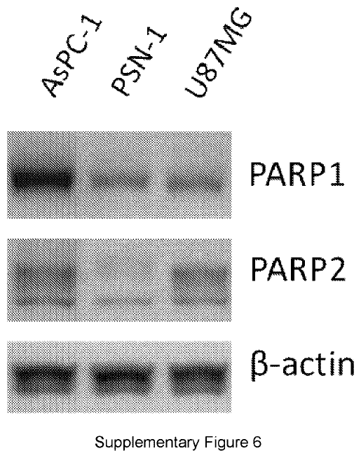

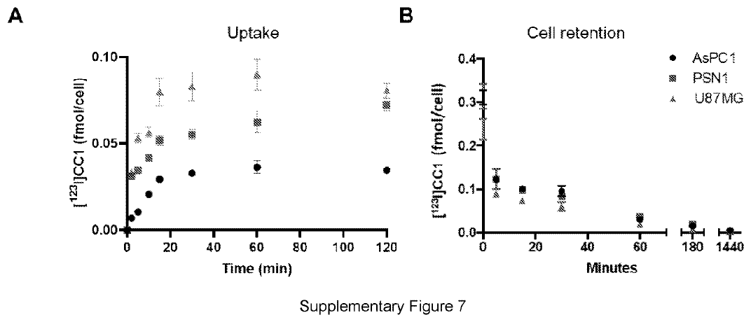

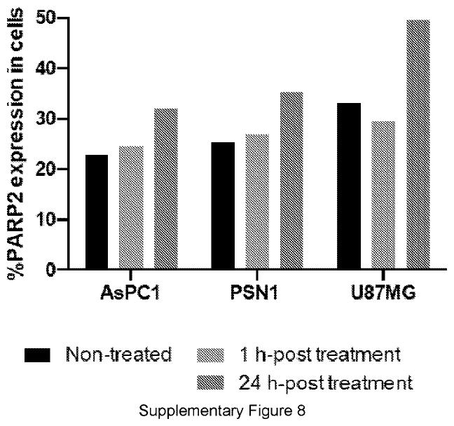

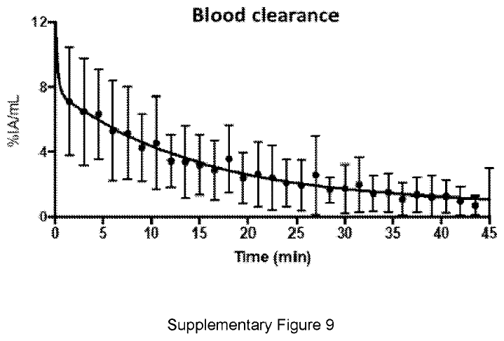

Supplementary FIG. 5: VOI analysis of dynamic imaging following intravenous administration of [¹²³I]CC1 in mouse.

### MATERIALS AND METHODS

### Synthesis and Characterisation of CC1

Unless otherwise noted, all reagents were purchased from Sigma-Aldrich and used without further purification. Olaparib, rucaparib, veliparib, talazoparib, and niraparib were purchased from Stratech Scientific Ltd (UK). Daidzin and Mycophenolate mofetil were purchased from Cambridge Bioscience Ltd (UK) and Insight Biotechnology Ltd, respectively. A modified literature procedure was used to synthesize CC1 (4-[(3-[(4-cyclopropylcarbonyl)piperazin-1-yl]carbonyl-4-iodophenyl]methyl(2H)phthalazin-1-one) (1). See Supplemental Information for a full description of protocols.

A commercially available colorimetric assay (Trevigen, Gaithersburg, MD) was used to measure PARP-1, 2 and 3 catalytic activity in vitro in the presence of varying concentrations of established PARP inhibitors or CC1, according to the manufacturer's instructions. The ability of 10 nM CC1 to inhibit the catalytic activity of a range of PARP family enzymes was performed by AMS Biotechnology Europe Ltd. according to BPS assay kit protocols.

### Radiosynthesis of [¹²³I]CC1

Sodium [¹²³I]iodide in 0.1 N NaOH (GE, UK). [¹²³I]CC1 was synthesised from the appropriate boronic pinacol ester precursor via a procedure modified from (1,2) (FIG. 1). Radiochemical yields of >95%, and a molar activity of >20 GBq/μmol were obtained routinely. Activity yield was >75% (n.d.c.), over a two-hour synthesis. See Supplemental Information for a full description of synthetic methodology.

### Cell Culture

Human malignant glioma (U87MG) cells were kindly donated by Prof. Nicola Sibson at our Institute, and maintained in high glucose Dulbecco's Modified Eagle Medium (DMEM) supplemented with 10% foetal bovine serum (FBS, Gibco), 2 mM L-glutamine, 100 units/mL penicillin, and 0.1 mg/mL streptomycin (Gibco). Pancreatic adenocarcinoma (AsPC1 and PSN1) cells were purchased from ATCC (UK), and maintained in Roswell Park Memorial Institute (RPMI) supplemented with 10% foetal bovine serum (FBS, Gibco), 2 mM L-glutamine, 100 units/mL penicillin, and 0.1 mg/mL streptomycin (Gibco). Cells were grown under a humidified environment at 37° C. and 5% CO₂. Cells were harvested and passaged using Trypsin-EDTA solution. Cells were used no more than 25 passages following resuscitation from liquid nitrogen storage. All cells were authenticated by STR profiling, and tested regularly for the absence of mycoplasma.

### PARP Protein Expression In Vitro

Relative expression of PARP isoforms in AsPC1, PSN1 and U87MG cells were determined by flow cytometry of live cells. AsPC1, PSN1 and U87MG cells were seeded separately in 96-well plates (1×10⁵ cells/well). Cells were washed with FACS buffer (PBS, 2% FBS, 1 mM EDTA, 0.1% NaN₃) through centrifugation at 500×g for 5 min. Immunostaining was performed using the Foxp3/transcription factor staining buffer set (eBioscience™, USA). Intracellular staining was conducted in permeabilization buffer for 30 min at 4° C. in the dark using the following antibodies separately: AF488-conjugated anti-PARP-1 (1:100; sc-80070), AF594-conjugated anti-PARP-2 (1:100; sc-393310) and AF488-conjugated anti-PARP-3 (1:100; sc-390771) from Santa Cruz Biotechnology Inc. (USA), AF488-conjugated anti-IMPDH2 (1:500; ab-200770) from Abcam plc. (UK). Fixable viability dye ef780 (1:4000; eBioscience™; 65-0865-14) was used to discriminate between live and dead cells. Fixation of immunostained cells was performed using 10% formaldehyde for 15 min at RT. Flow cytometry was conducted using a CytoFLEX flow cytometer (Beckman Coulter, USA), with appropriate lasers and filters, positive and negative controls. Data were analyzed using FlowJo™ (Tree Star Inc., BD Biosciences, USA).

### In Vitro Uptake and Specificity of [¹²³I]CC1

AsPC1 (1×10⁵ cells/well), PSN1 cells (7.5×10⁴ cells/well) or U87MG (1×10⁵ cells/well) were seeded separately in 24-well plates containing growth medium and allowed to adhere for at least 20 h. Cells were washed and exposed to cold, unlabelled PARP inhibitors (100 μM, in a total of 270 μL growth medium) for 45 min at 37° C. Then, [¹²³I]CC1 was added, and the cells incubated at 37° C. for a 45 min. Cell culture medium was removed, and cells washed with PBS. Cells were lysed using RIPA buffer (950 mM Tris pH 8.0, 1% NP40, 0.5% sodium deoxycholate, 0.1% sodium dodecyl sulphate, and 150 mM sodium chloride) for 15 min at room temperature, and the amount of ¹²³I in cell lysates was measured using an automated gamma counter (PerkinElmer). In a separate experiment, cells were prepared in a similar manner, but cells were washed and exposed to [¹²³I]CC1 at 37° C. for different intervals (1-120 min). The amount of ¹²³I in cell lysates was measured as above.

### Cell Retention Assay

Cells were seeded separately in 24-well plates as above, and allowed to adhere for at least 20 h. Cells were washed and exposed to [¹²³I]CC1 at 37° C. for 30 min. Cell culture medium was removed, washed with PBS, and provided with fresh medium (300 μL). Cells were then further incubated at 37° C. Cell culture medium was removed, and cells were washed with PBS at different time points (0-24 h). Cells were lysed using RIPA buffer (950 mM Tris pH 8.0, 1% NP40, 0.5% sodium deoxycholate, 0.1% sodium dodecyl sulphate, and 150 mM sodium chloride) for 15 min at room temperature, and the amount of ¹²³I in the cell lysates was measured using an automated gamma counter (PerkinElmer).

### Colony Formation Assay

Aliquots of cells (PSN1 and U87MG, 5000 cells) were exposed increasing amounts of [¹²³I]CC1 (0-100 kBq), or cold, unlabelled olaparib or CC1 (0-0.1 mM, in a total of 200 μL growth medium) for 60 min at 37° C. Separate aliquots of cells were irradiated (0-10 Gy, using a ¹³⁷Cs irradiator, 0.8 Gy/min). Cells were then washed, a fraction of which was then transferred to 6-well plates containing 2 mL of growth medium, and colony formation was measured two weeks after treatment. Colony numbers were normalized to the number of cells transferred to the plates to obtain plating efficiency, which was in turn normalized to that of untreated cells.

### Quantification of Nuclear Protein Expression Post [¹²³I]CC1 Treatment

AsPC1, PSN1 or U87MG (1×10⁶ cells/well in 2 mL growth medium) were seeded in 6-well plates and allowed to adhere for at least 20 h. Cells were washed and exposed to [¹²³I]CC1 (30 μL, 50 kBq, 20 GBq/μmol, in 2 mL of growth medium) at 37° C. for 1 h. Cells were harvested using trypsin-EDTA solution and washed with FACS buffer (PBS, 2% FBS, 1 mM EDTA, 0.1% NaN₃) and centrifuged at 500×g for 5 min. Immunostaining was performed using the Foxp3/transcription factor staining buffer set (eBioscience™, USA). Intracellular staining was conducted in permeabilization buffer for 30 min at 4° C. in the dark using the following antibodies separately: AF488-conjugated anti-PARP-1 (1:100; sc-80070), AF594-conjugated anti-PARP-2 (1:100; sc-393310) and AF488-conjugated anti-PARP-3 (1:100; sc-390771) from Santa Cruz Biotechnology Inc. (USA), AF488-conjugated anti-IMPDH2 (1:500; ab-200770) from Abcam plc. (UK), yH2AX (1:1000; JBW301 Merck-Millipore). Fixable viability dye ef780 (1:4000; eBioscience™; 65-0865-14) was used for live/dead discrimination. Fixation of immunostained cells was performed for 15 min at RT. Flow cytometry was conducted using a CytoFLEX flow cytometer (Beckman Coulter, USA), with appropriate laser and filters, positive and negative controls. Data were analyzed using FlowJo™ (Tree Star Inc., BD Biosciences, USA).

### SPECT/CT Imaging and Biodistribution of [¹²³I]CC1 in Xenograft-Bearing Immunocompromised Mice

Female Balb/c nu/nu (OlaHsd-Foxn1^{nu}) mice, aged 4-6 weeks were purchased from Envigo (UK). Animals were housed in IVC cages, up to 6 per cage, in an artificial day-night cycle facility. Food and water were provided ad libitum.

PSN1 cells were harvested using trypsin, washed twice with PBS, and reconstituted in PBS: Matrigel® Matrix High Concentration (1:1). Cell suspensions (PSN1: 2×10⁶ cells/100 μL cells/100 μL) were injected subcutaneously in the front right flank of the mice, and allowed to grow to a size of approximately 200 mm³.

Animals were administered [¹²³I]CC1 (3 MBq in 100 μL of PBS, Aₘ=20 GBq/μmol) by intravenous injection via the lateral tail vein. To evaluate the specificity of tumour uptake, an excess of unlabelled rucaparib (0.5 mg) was co-administered as blocking agent. Either after 60 or 120 min after radiolabelled compound injection, animals were euthanised. Selected organs, tissues and blood were removed, and the percentage of the injected dose per gram of tissue (% ID/g) was determined, using a HIDEX automated gammacounter.

Localisation of [¹²³I]CC1 in PSN1 xenografts was determined ex vivo using autoradiography performed on frozen tumour sections (10 μm). Uptake in PSN1 tumours was further compared to immunohistochemistry, staining for PARP1, 2, and 3, and γ-H2AX. Full details of procedures and protocols have been provided in the Supplemental Information.

Separately, animals were administered [¹²³I]CC1 and dynamic PET images were acquired over 45 minutes. Images were analysed using VOI-based analysis, using PMOD.

### In Vivo Therapy Studies

PSN1 xenograft-bearing mice, with average tumour sizes of 82 mm³, were randomly grouped into cohorts, and intravenously injected with [¹²³I]CC1 (3 MBq in 100 μL of PBS, Aₘ=20 GBq/μmol) or an equivalent amount of cold, unlabeled CC1. Mice and tumour sizes were monitored daily. Study endpoint was determined based humane endpoints, including a tumour size >1200 mm³, or weight loss >15%.

### Statistical Analyses

All data were obtained at least in triplicate. All statistical analyses and nonlinear regression were performed using GraphPad Prism v6 or higher (GraphPad Software, San Diego, CA, USA). Data were tested for normality and analysed as appropriate by 1 or 2-way analysis (ANOVA). Results are reported as mean±one standard deviation, unless stated otherwise.

### Results

### CC1 is a Selective and Potent PARP Inhibitor

CC1 proved to be a potent PARP inhibitor, with cell-free IC50 values, determined in-house, of 2.9 and 0.6 nM for PARP 1 and 2, respectively (FIG. 1B, Supplementary FIG. 1). This compared favorably to olaparib (2.6 and 0.8 nM, in the same assay). Contrarily, PARP3 inhibition was lower for CC1 (68 nM) compared to olaparib (18 nM). A single dose of CC1 (10 nM) was able to inhibit PARP1, PARP2, and PARP6 (FIG. 1B), but to a lesser degree compared to olaparib, rucaparib, and other established PARP inhibitors. If interpreted as a proxy for PARP binding, CC1 binds PARP1, PARP2, and PARP3.

### [¹²³I]CC1 Uptake In Vitro

AsPC1, PSN1, and U87MG cells expressed PARP1, 2, and 3 to varying degrees with PARP1 expression AsPC1>U87MG>PSN1. [¹²³I]CC1 was taken up in all three cell lines within minutes, plateauing after 1 h, whereas [¹²³I]CC1 was retained briefly in cells (Supplementary FIG. 3). [¹²³I]CC1 was taken up selectively in AsPC1, PSN1 and U87MG cells, with uptake of around 0.3 pmol/cell (FIG. 1D). Addition of cold, non-labelled PARP inhibitors reduced uptake significantly, proving PARP selectivity (FIG. 1E). Uptake of [¹²³I]CC1 was also reduced by addition of daidzin and m.mofetil, two drugs binding other NAD⁺-binding enzymes, ALDH2 and IMPDH2, respectively. Interestingly, exposure of all three cell types to [¹²³I]CC1 resulted in increased expression of PARP1 and PARP2, 24 h later (FIG. 4). In addition, yH2AX expression, a marker of DNA double strand breaks, increased significantly 24 h after exposure to [¹²³I]CC1.

### CC1 Reduces Survival in a Colony Formation Assay

Clonogenic survival of cells was significantly reduced by exposure to relatively small amounts of [¹²³I]CC1, from amounts as small as 10 Bq for U87MG cells. IC50 values for [¹²³I]CC1 equate 1 kBq/mL for PSN1 cells. Equivalent amounts of CC1 or olaparib had no effect.

### Biodistribution of [¹²³I]CC1

Biodistribution of [¹²³I]CC1 (3 MBq, 20 GBq/μmol) was investigated in PSN1 xenograft-bearing mice (FIG. 5). High uptake in liver, gallbladder, large/small intestine, showing a similar hepatobiliary clearance pattern as [¹⁸F]olaparib in the same model (1). Blood clearance showed a two-phase decay with a fast and slow halflife of 16.85 and 1.35 min, resulting in a weighed halflife of 1.86 min (CI95 1.64-2.09 min). Tumour uptake in PSN-1 xenografts amounted to 0.9±0.06% ID/g at 1 h after intravenous administration.

### Therapeutic Efficacy of [¹²³I]CC1 in a PSN1 Mouse Model

Application of [¹²³I]CC1 for treatment of PARP enzyme expressing tumours was tested using PSN1-xenograft bearing mice. A single intravenous administration of [¹²³I]CC1 led to significant inhibition of tumour growth compared to animals exposed to cold, unlabelled CC1 (p=0.04), without signs of gross toxicity, as determined by a lack of weight loss of the mice.

### DISCUSSION

PARP inhibitors trap PARP enzyme to broken DNA. This close proximity to DNA makes PARP inhibitors a good vehicle to carry for Auger electron emitting radionuclides. These short-range electron emitting radionuclides are significantly radiotoxic to cells, if decaying closely to DNA. Here, we synthesised a compound, containing ¹²³I, an Auger electron emitter, that binds PARP. This type of compound can be employed for therapeutic advantage. Importantly, the very low concentrations that already allow radionuclide therapy would not lead to an overall catalytic inhibition of PARP enzyme activity. Contrarily, it is the Auger electron emission that cause DNA damage, causing growth inhibition. We found that [¹²³I]CC1 binds specifically to PARP enzyme, and significantly lowers clonogenic survival of PARP-expressing cancer cells after being exposed for one hour only to as little as 10 Bq [¹²³I]CC1 onwards, a very low amount. Interestingly, because ¹²³I, apart from being an Auger electron emitter, also emits gamma rays, that allow detection by gamma-detector, and SPECT imaging. We found that a dose of [¹²³I]CC1 necessary for imaging was already effective in inhibiting tumour growth in a human tumour xenograft mouse model. However, other radiolabelled compounds, such as [¹⁸F]olaparib, may be used as a companion diagnostic. Specifically. [¹⁸F]olaparib, can be used for PET imaging of PARP, and act as a companion diagnostic.

Gross toxicity in vivo was not observed: uptake in liver, gall bladder and bowel, as well as kidneys for other radiolabelled PARP inhibitors was assumed to be luminal. The short pathlength of Auger electrons ensures that very little DNA damage is inflicted by the radiolabelled compound in those tissues.

### EXAMPLES—PART B

### Figure Legends

FIG. 6: Blocking of [¹²³I]CC1 uptake in U87MG cells by CC1 in PSN1, AsPC1, or CAPAN1 cells. Asterisks indicate levels of statistical significance: *, P<0.05.

FIG. 7: (E) Autoradiography of tumour sections harvested 2 h following intravenous injection of [¹²³I]CC1 (3 MBq) in mice bearing PSN-1, U87MG, or MDA-MB-231 xenografts. (F) Tumour uptake 2 h after intravenous injection of [¹²³I]CC1 (3 MBq) in mice bearing PSN-1, U87MG, or MDA-MB-231 xenografts. Asterisks indicate levels of significance: ns, P>0.05; *, P<0.05.

FIG. 8: (A, B) Tumour growth and Kaplan-Meier survival plots following a single intravenous administration of [¹²³I]CC1 (3 MBq, 124.2-341.9 GBq/μmol) or an equivalent amount of non-labelled CC1 in mice bearing PSN-1 xenografts. (C)Representative photographs of mice bearing PSN-1 xenografts, 15 days following [¹²³I]CC1 or CC1 administration (D) Animal weights following administration of [¹²³I]CC1 or CC1. (E, F) Tumour growth curves following a single intravenous administration of [¹²³I]CC1 (3 MBq) or an equivalent amount of non-labelled CC1 in mice bearing U87MG or MDA-MB-231 xenografts. (G) Representative H&E staining in selected normal tissues at 28 days following administration of [¹²³I]CC1 (3 MBq) or untreated C57/Bl6 mice.

### SUPPLEMENTARY FIGURES

Supplementary FIG. 6: Western blot showing PARP1 and PARP2 expression in AsPC1, PSN-1, and U87MG in vitro whole cell lysates.

Supplementary FIG. 7: (A) Time dependent uptake of [¹²³I]CC1 in AsPC1, PSN1 and U87MG cells. (B) Cell-associated retention of [¹²³I]CC1, after a 1-hour exposure of AsPC1, PSN1 and U87MG cells.

Supplementary FIG. 8: Flow cytometry measurements show increased numbers of cells with higher PARP2 expression, 1 or 24 h after a 1-hour exposure to [¹²³I]CC1 (50 KBq. 18 GBq/μmol) in PSN1, AsPC1, and U87MG cells.

Supplementary FIG. 9: VOI analysis of blood clearance following intravenous bolus administration of [¹²³I]CC1 (3 MBq) to PSN1 xenografts bearing-mice.

### Materials and Methods

Synthesis and characterisation of CC1, as well as radiosynthesis of [¹²³I]CC1 were conducted as described in part A hereinbefore.

### Cell Culture

Human malignant glioma (U87MG) cells were kindly donated by Prof. Nicola Sibson at our Institute and maintained in high glucose Dulbecco's Modified Eagle Medium (DMEM) supplemented with 10% foetal bovine serum (FBS, Gibco), 2 mM L-glutamine, 100 units/mL penicillin, and 0.1 mg/mL streptomycin (Gibco). Pancreatic adenocarcinoma (AsPC1, PSN1, and CAPAN-1) and breast cancer (MDA-MB-231) cells were purchased from ATCC (UK), and maintained in Roswell Park Memorial Institute (RPMI) supplemented with 10% foetal bovine serum (FBS, Gibco), 2 mM L-glutamine, 100 units/mL penicillin, and 0.1 mg/mL streptomycin (Gibco). Cells were grown under a humidified environment at 37° C. and 5% CO₂. Cells were harvested and passaged using trypsin-EDTA solution. Cells were used for no more than 20 passages following resuscitation from liquid nitrogen storage. All cells were authenticated by the provider and STR profiling and tested regularly for the absence of mycoplasma.

### PARP Protein Expression In Vitro

Relative expression of PARP1, 2, and 3 were determined by flow cytometry of live cells as described in part A hereinbefore. In a separate study, DNA double strand break damage was quantified using an anti-γH2AX antibody (JBW301, 1:800), in combination with an AF488-conjugated anti-mouse antibody (Santa Cruz Biotechnology Inc.). Fixable viability dye ef780 (1:4000; eBioscience™; 65-0865-14) was used to discriminate between live and dead cells. Fixation of immunostained cells was performed using the Foxp3 Transcription Factor Staining Buffer Set (eBioscience™, 00-5523-00) for 15 min at RT. Flow cytometry was conducted using a CytoFLEX flow cytometer (Beckman Coulter, USA), with appropriate lasers and filters, positive and negative controls. Data were analyzed using FlowJo™ (Tree Star Inc., BD Biosciences, USA).

### In Vitro Uptake and Specificity of [¹²³I]CC1

AsPC1 (1×10⁵ cells/well), PSN1 (7.5×10⁴ cells/well) or U87MG (1×10⁵ cells/well) were seeded as described in part A hereinbefore. CAPAN-1 cells (7.5×10⁴ cells/well) were seeded following the same protocol described in part A hereinbefore. In a separate experiment, cells were exposed to [¹²³I]CC1 for 30 min at 37° C., washed, supplied with fresh growth medium, and the amount of ¹²³I associated with cells measured after varying intervals as described in part A hereinbefore.

### Colony Formation Assay

Following the protocol outlined in part A hereinbefore, colony formation assays were conducted on aliquots of PSN1, U87MG and MDA-MB-231 cells.

### Quantification of Nuclear Protein Expression Post [¹²³I]CC1 Treatment

Quantification of AsPC1, PSN1 or U87MG cells was conducted following the protocol outlined in part A hereinbefore. Relative expression of PARP1 and 2 was measured using flow cytometry. γH2AX expression, as a measure for DNA double strand break damage, was assessed in a similar manner.

### SPECT/CT Imaging and Biodistribution of [¹²³I]CC1 in Xenograft-Bearing Immunocompromised Mice

Following the protocol described in part A hereinbefore, PSN1, U87MG, or MDA-MB-231 cells were harvested using trypsin, washed twice with PBS, and reconstituted in PBS: Matrigel® Matrix High Concentration (1:1). Cell suspensions were injected subcutaneously in the hind right flank and allowed to form tumours. When tumours reached 100-200 mm³, animals were administered [¹²³I]CC1 (3 MBq in 100 μL of PBS, Aₘ=124.3 GBq/μmol) by intravenous bolus injection via the lateral tail vein. To evaluate the specificity of tumour uptake, an excess of unlabelled rucaparib (0.5 mg) was co-administered as a blocking agent in some animals. Either 60 or 120 min after radiolabelled compound injection, animals were euthanised (n=3 per group). Selected organs, tissues and blood were removed, and the percentage of the injected activity per gram of tissue (% IA/g) was determined, using a HIDEX automated gammacounter.

In a selected number of animals (n=3), animals were anaesthetised using 2% isoflurane, and dynamic SPECT/CT imaging was performed over 1 h, using a MILabs VECTor4 camera, equipped with an ultra-high-resolution rat/mouse collimator (1.8 mm), followed by a cone-beam CT scan (55 kV, 0.19 mA) for anatomical reference and attenuation correction. Anesthesia was maintained using 2.5% isoflurane throughout the duration of image acquisition. SPECT images were reconstructed using U-SPECT-Rec3.22 software (MILabs, Utrecht, The Netherlands), applying a pixel-based algorithm, ordered subset expectation maximisation (OSEM) with 4 subsets, 4 iterations and 0.6 mm voxel size for lodine-123 (energy window settings 141.3-172.7 keV). Reconstructed SPECT and CT images were viewed and analyzed using PMOD v.3.37 (PMOD Technologies, Zurich, Switzerland).

Localisation of [¹²³I]CC1 in PSN1 xenografts was further determined ex vivo using autoradiography performed on frozen tumour sections (10 μm). Uptake in tumour tissue was further compared to immunohistochemistry, staining for PARP1 and 2.

### Toxicology

H&E staining was performed on selected tissues at 24 h and 28 days following intravenous administration of 3 MBq [¹²³I]CC1 to otherwise naïve C57/Bl6 mice (n=3 per time point). Liver, spleen, kidneys and intestines were investigated by an experienced veterinary pathologist and compared to age-matched non-treated control animals.

Tissues were further stained for γH2AX to investigate DNA double strand break damage, and livers were additionally stained for PAS, as a marker for glycogen storage.

### In Vivo Therapy Studies

PSN1 xenograft-bearing mice, with average tumour sizes of 82 mm³, were randomly grouped into cohorts, and intravenously injected with [¹²³I]CC1 (3 MBq in 100 μL of PBS, Aₘ=124-342 GBq/μmol) or an equivalent amount of unlabeled CC1. Mice were monitored daily. Tumour sizes were determined by calliper. Study endpoints were based on humane endpoints, including a tumour size >1000 mm³, or weight loss >15%.

### Dosimetry

In vitro, radiation absorbed dose was determined using the MIRD Cell package employing uptake and retention data in U87MG cells. Cell and cell nuclear dimensions were approximated as concentric circles, of sizes based on confocal microscopy (14 μm cell diameter, 8 μm nucleus diameter).

### Statistical Analyses

All data were obtained at least in triplicate. All statistical analyses and nonlinear regression were performed using GraphPad Prism v8 or higher (GraphPad Software, San Diego, CA, USA). Data were tested for normality and analysed as appropriate by 1 or 2-way analysis (ANOVA). Results are reported as mean±one standard deviation, unless stated otherwise.

### Results

### [¹²³I]CC1 Uptake in Cells In Vitro is PARP-Selective

[¹²³I]CC1 was produced reliably, in good radiochemical yield, with high Aₘ (FIG. 1D). AsPC1, PSN1, and U87MG cells expressed PARP1, and 2 to varying degrees with PARP1 expression AsPC1>U87MG>PSN1 (Supplementary FIG. 6). [¹²³I]CC1 was taken up in all three cell lines within minutes, plateauing after 1 h (Supplementary FIG. 7A). [¹²³I]CC1 was retained briefly in cells (Supplementary FIG. 7B). [¹²³I]CC1 was taken up selectively in AsPC1, PSN1, and U87MG cells, with uptake of around 0.3 fmol/cell (FIGS. 1E, 1F and 6). Addition of structurally related and unrelated non-labelled PARP inhibitors significantly reduced the cell-associated amount of [¹²³I]CC1 in all cell lines (P<0.0001), suggesting PARP-selective binding of [¹²³I]CC1 (FIG. 6).

### [¹²³I]CC1 Caused DNA Damage, and Reduces Clonogenic Survival

γH2AX expression, a marker of DNA double strand breaks, increased markedly 24 h after a one-hour exposure of PSN-1 and U87MG cells to a small amount of [¹²³I]CC1 (50 kBq) (FIG. 2B). Exposure of all three cell types to [¹²³I]CC1 resulted in increased expression of PARP1 and PARP2, 24 h after a one-hour exposure (FIG. 2A and Supplementary FIG. 8). In vitro cell dosimetry, calculated by MIRDcell, based on uptake and retention values (Supplementary FIG. 7), estimated the radiation absorbed dose (over 24 hours) to 7.89 Gy.

Clonogenic survival of cells was significantly reduced by exposure to relatively small amounts of [¹²³I]CC1, from amounts as small as 10 Bq (20 MBq/μmol) for U87MG cells. IC50 values for [¹²³I]CC1 equated 631±35 Bq (in 200 μl) for U87MG cells. Efficacy in PSN1 cells in vitro was far less pronounced. Equivalent amounts of CC1 or olaparib had no effect on clonogenic survival (FIGS. 1E and 1F).

### Biodistribution of [¹²³I]CC1 Shows a Hepatobiliary Clearance Pattern

Dynamic SPECT/CT imaging and biodistribution of [¹²³I]CC1 (3 MBq, 20 GBq/μmol) was investigated in PSN1 xenograft-bearing mice (FIGS. 3, 4, 7 and Supplementary FIGS. 5 and 9). High uptake in liver and intestines indicated a hepatobiliary clearance pattern, similar to other radiolabelled PARP inhibitors (FIGS. 3A and B). Blood clearance showed a two-phase decay with a fast and slow half-life of 16.85 and 1.35 min, resulting in a weighed half-life of 1.86 min (CI95 1.64-2.09 min) (Supplementary FIG. 9B). Cut- and -count biodistribution studies showed tumour uptake in PSN-1 xenografts amounted to 0.9±0.06% IA/g at 1 h after intravenous administration, and normal issue uptake in line with dynamic SPECT imaging (FIG. 3C). After 24 h, 0.033±0.017% IA/g of [¹²³I]CC1 remained in the tumour 24 h after injection. Co-administration of an excess of rucaparib, to block the NAD+ binding pocket in the PARP enzymes, significantly reduced tumour uptake (P<0.05) (FIG. 4A), as confirmed by autoradiography (FIG. 7E). Tumour uptake in U87MG and MDA-MB-231 xenografts was significantly lower compared to PSN-1 xenografts (0.46±0.01% IA/g for U87MG and 0.19±0.01% IA/g for MDA-MB-231 xenografts) (FIGS. 7E and 7F).

### CC1 Displays Limited Normal Tissue Toxicity

With a view to using [¹²³I]CC1 for radionuclide therapy of tumours, an evaluation into whether the radiolabelled compound induced any toxicity in normal tissue was conducted. Based on its biodistribution pattern, radiation-induced damage from exposure to [¹²³I]CC1 may be expected in liver, and intestines. Due to partial renal clearance, kidneys were also evaluated, and given the ability of the radiolabeled PARP inhibitor [¹⁸F]olaparib to bind specifically to splenic tissue, the spleen was also considered (FIG. 8).

Intestines of mice administered [¹²³I]CC1 (3 MBq) intravenously showed minimal proprial infiltration by lymphocytes and plasma cells. Scattered intact eosinophils were present within the propria, but enterocytes of none of the mice showed signs of generation or necrosis, with apical brush borders remaining intact. Mitotic figures were regularly present and within normal counts. Observations were no different at 24 h or 28 days after administration. The kidneys showed no observable changes, while in the spleen, mild to moderate numbers of hemosiderophages were observed at 24 h and 28 days after administration alike. No signs of necrosis were seen.

In the liver, hepatocellular nuclei were centrally located and showed no signs of necrosis. Some small foci of extramedullary hematopoiesis were present. Mild anisocytosis and anisokaryosis were seen. A few individual scattered hepatocytes (0.1-0.2 per field) showed a shrunken shape, hypereosinophilic cytoplasm and a shrunken nucleus with condensed chromatin, interpreted as pyknosis. The one marked effect in the liver consisted of diffuse cytoplasmic pallor/rarefaction, created by optically empty feathery spaces and vacuoles and some remaining floccular granulated cytoplasmic material, often peripheralised. Effects were slightly more pronounced in animals 28 days after administration of [¹²³I]CC1, although not statistically significantly so.

### Potent Tumour Growth Inhibition by [¹²³I]CC1 in PDAC-Xenograft Tumour Bearing Mice

Intravenous administration of relatively small amounts of [¹²³I]CC1 (3 MBq) showed significant tumour growth delay in PSN1-xenograft bearing mice (FIG. 8). A single intravenous administration of [¹²³I]CC1 led to significant inhibition of tumour growth compared to animals exposed to cold, unlabelled CC1 (p=0.04), without signs of gross toxicity, as determined by a lack of weight loss of the mice (p>0.05). U87MG xenografts responded less than PSN-1 xenografts (p>0.05), whereas MDA-MB-231 xenografts were not affected in their growth by the same amount of [¹²³I]CC1 (p>0.05).

### DISCUSSION

[¹²³I]CC1 has been shown to bind selectively to PARP, causing DNA double strand break damage, and reducing clonogenic survival in vitro and tumour growth in vivo. ¹²³I—CC1 also induced increased expression of PARP1 and PARP2 in tumour tissue. Although this may form the basis of a possible feedback mechanism for multiple administrations, this was not investigated since a single administration of ¹²³I—CC1 (3 MBq) was therapeutically efficacious in PSN-1 xenograft-bearing mice.

Although efficacy in PSN1 cells in vitro was less pronounced than the in vitro response in U87MG cells, in vivo uptake in PSN1 tumours was higher than that in U87MG tumour xenografts, resulting in better therapeutic efficacy. This could be due to higher uptake of ¹²³I resulting in higher radiation absorbed dose, differential radiation sensitivity in vitro vs. in vivo, and/or differences in growth rate and repopulation in vitro vs. in vivo.

Compared to ¹²³I-MAPi, ^{123/125}I—KX1, ¹²⁵I—KX-02-019, ²¹¹At-MM4, ¹²⁵I-PARP-01 and ⁷⁷Br-GD1, [¹²³I]CC1 has been shown to be effective after a single intravenous administration, at relatively low doses. It has already been shown in previous work that small changes in the structure of PARP inhibitors will give rise to major changes in their characteristics. Factors affecting the efficacy of radioligand therapy with radiolabelled PARP inhibitors may include binding affinity, binding spectrum, bioavailability, pharmacokinetics, and tumour uptake. Additionally, trapping of PARP enzyme by PARP inhibitors, and therefore by radiolabelled PARP inhibitors may play a significant role in their efficacy. Therefore, it can be expected that different radiolabelled PARP inhibitors may have very different therapeutic indices.

Many studies with radiolabelled PARP inhibitors incorporate Auger electron emitters, given trapping of the PARP enzyme brings the PARP inhibitors in very close proximity to the DNA, an excellent match with the short range of Auger electron emitters. Even though ¹²⁵I is a very efficient Auger electron emitter, with some 23 low-energy electrons emitted per decay, it is preferred to use ¹²³I, taking into account half-life considerations. ¹²³I has 14 AEs per decay, a more manageable half-life and has the advantage of being available worldwide.

Normal tissue toxicity was minimally affected. The lack of normal tissue toxicity may be explained by the very short range of ¹²³I's AEs: Despite high uptake in gall bladder, intestines and urinary bladder, because of the luminal uptake of PARP inhibitors in these organs, radiation dose from Auger electron emissions will be minimal. In the liver, the uptake of another labelled PARP inhibitor was shown to be cytoplasmic, and not nuclear. Given that Auger electron emitters decaying in the cytoplasm are 30 times less cytotoxic compared to those decaying in the nucleus, cytoplasmic uptake of [¹²³I]CC1 in the liver would be far less cytotoxic.

It has already been shown that uptake of an ¹⁸F-labelled olaparib in tumour tissue was much dependent on the administered mass, and molar activity. It is likely that this is also the case for ¹²³I—CC1, and other PARP inhibitor-based radioligand therapies.

The gamma emissions from ¹²³I also make it an imaging agent. Therefore, [¹²³I]CC1 may be considered a true theragnostic agent, with lower administered doses used for SPECT imaging, and larger doses for therapy. Alternatively, ¹⁸F-(6), or even ¹²⁴I-labelled variants may be used to gauge the relative expression of the target enzyme in tumour tissue, by PET imaging.

Taken together, [¹²³I]CC1 is a promising Auger-electron emitting PARP-inhibitor based therapeutic radiopharmaceutical.

1. A compound of formula I, or a pharmaceutically acceptable salt or solvate thereof: 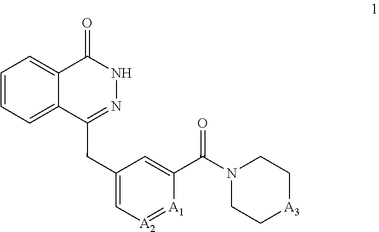
wherein:
A₁ and A₂ are selected from CR₁ and CH, CR₁ and CF, CR₁ and N, CH and CR₁, CF and CR₁ or NH and CR₁ respectively;
R₁ is a radioisotope of iodine (I) or astatine (At);
A₃ is selected from N—X₁—R₃ or C(R₄)OR₅;
X₁ is absent or selected from —C(O)—, —[CH₂]₁₋₃—, —C(O)NH—, —C(O)O—, or —S(O)₀₋₂—;
R₃ is H, (1-10C)alkyl, (3-8C)cycloalkyl, aryl, heterocyclyl or heteroaryl, wherein each alkyl, cycloalkyl, aryl, heterocyclyl or heteroaryl group is optionally substituted by one or more R_{A} substituents;
R₄ is selected from H, or C₁₋₄ alkyl; and
R₅ is selected from (1-7C)alkyl, (3-8C)cycloalkyl, heterocyclyl and aryl, which groups are optionally substituted by one or more R_{A} substituents;
or R₄ and R₅ together with the carbon and oxygen atoms to which they are attached form a spiro-C₅₋₇ oxygen-containing heterocyclyl, which is optionally substituted by one or more R_{A} substituents or fused to a phenyl or 5 or 6 membered heteroaryl ring; and
each R_{A} substituent is independently selected from fluoro, cyano, (1-4C)alkyl, (1-4C)haloalkyl, (1-4C)hydroxyalkyl, phenyl, 4 to 6 membered heterocyclyl, 5 to 6-membered heteroaryl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), —SO₂N(R_{1b})R₁ₐ, —N(R_{1b})SO₂R₁ₐ wherein R₁ₐ and R_{1b} are each independently selected from hydrogen, (1-4C)alkyl or (3-6C)alkyl.

2. A compound according to claim 1, or a pharmaceutically acceptable salt or solvate thereof, wherein:
(i) A₁ is CR₁ and A₂ is selected from CH, CF, or N; or
(ii) A₁ is selected from CH, CF, and N, and A₂ is CR₁.

3. A compound according to claim 2, or a pharmaceutically acceptable salt or solvate thereof, wherein A₁ is CR₁ and A₂ is CH.

4. A compound according to any one of claims 1 to 3, or a pharmaceutically acceptable salt or solvate thereof, wherein R₁ is a radioisotope of iodine (I) is selected from ¹²³I, ¹³¹I, ¹²⁷I or ¹²⁴I, or a radioisotope of astatine (At) which is ²¹¹At.

5. A compound according to claim 1 or claim 2, or a pharmaceutically acceptable salt or solvate thereof, wherein R₁ is a radioisotope of iodine (I) is selected from ¹²³I, ¹²⁴I or ¹³¹I.

6. A compound according to claim 1 or claim 3, or a pharmaceutically acceptable salt or solvate thereof, wherein R₁ is ¹²³I.

7. A compound according to any one of the preceding claims, or a pharmaceutically acceptable salt or solvate thereof, wherein A₃ is N—R₃.

8. A compound according to any one of the preceding claims, or a pharmaceutically acceptable salt or solvate thereof, wherein R₃ is (1-6C)alkyl, (3-6C)cycloalkyl, phenyl, a 4 to 6-membered heterocyclyl or a 5 to 6-membered heteroaryl, each of which is optionally substituted by one or more R_{A} substituents.

9. A compound according to any one of the preceding claims, or a pharmaceutically acceptable salt or solvate thereof, wherein R₃ is (1-6C)alkyl or (3-6C)cycloalkyl, each of which is optionally substituted by one or more R_{A} substituents.

10. A compound according to any one of the preceding claims, or a pharmaceutically acceptable salt or solvate thereof, wherein R₃ is (1-6C)alkyl or (3-6C)cycloalkyl.

11. A compound according to any one of the preceding claims, or a pharmaceutically acceptable salt or solvate thereof, wherein R₃ is (3-6C)cycloalkyl.

12. A compound according to any one of the preceding claims, or a pharmaceutically acceptable salt or solvate thereof, wherein R₅ is cyclopropyl.

13. A compound according to any one of claims 1 to 6, or a pharmaceutically acceptable salt or solvate thereof, wherein A₃ is C(R₄)OR₅.

14. A compound according to claim 13, or a pharmaceutically acceptable salt or solvate thereof, wherein R₄ is selected from H, C₁₋₄ alkyl and R₅ is selected from (1-7C)alkyl, 4 to 6-membered heterocyclyl and phenyl, which groups are optionally substituted by one or more R_{A} substituents.

15. A compound according to claim 13, or a pharmaceutically acceptable salt or solvate thereof, wherein R₄ is selected from H and R₅ is (1-4C)alkyl.

16. A compound according to claim 13, or a pharmaceutically acceptable salt or solvate thereof, wherein R₄ is selected from H and R₅ is methyl.

17. A compound according to any one of the preceding claims, or a pharmaceutically acceptable salt or solvate thereof, wherein R_{A} is selected from:
(i) fluoro, cyano, (1-4C)alkyl, (1-4C)haloalkyl, (1-4C)hydroxyalkyl, phenyl, 4 to 6 membered heterocyclyl, 5 to 6-membered heteroaryl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), —SO₂N(R_{1b})R₁ₐ, —N(R_{1b})SO₂R₁ₐ, wherein R₁ₐ and R_{1b} are each independently selected from hydrogen or (1-4C)alkyl; or
(ii) fluoro, cyano, (1-4C)alkyl, (1-4C)haloalkyl, (1-4C)hydroxyalkyl, —OR₁ₐ, —NR₁ₐR_{1b}, —C(O)R₁ₐ, —C(O)N(R_{1b})R₁ₐ, N(R_{1b})C(O)R₁ₐ, —S(O)ₚR₁ₐ (where p is 0, 1 or 2), —SO₂N(R_{1b})R₁ₐ, —N(R_{1b})SO₂R₁ₐ, wherein R₁ₐ and R_{1b} are each independently selected from hydrogen or (1-4C)alkyl.

18. A compound according to any one of the preceding claims, or a pharmaceutically acceptable salt or solvate thereof, wherein the compound is a compound of formula Ia, Ib, Ic, Id, Ie, If, Ig, Ih, Ii, Ij or Ik shown below: 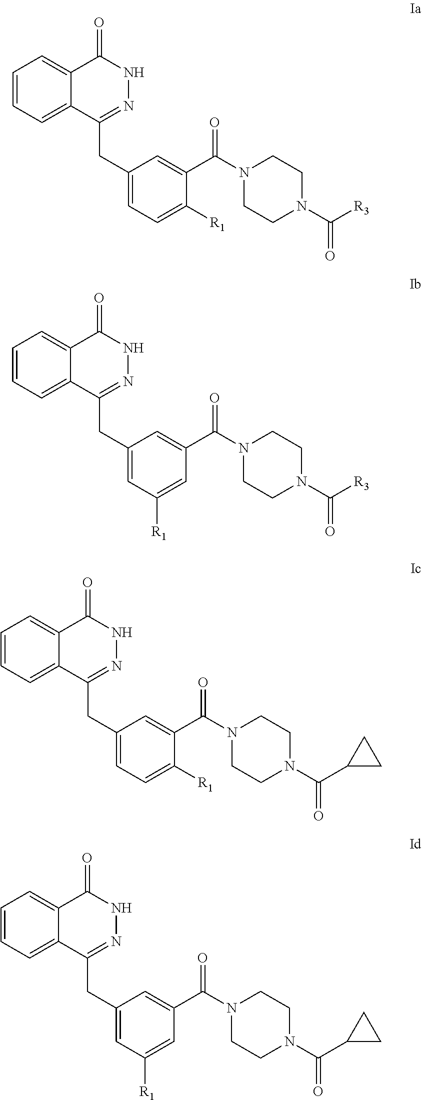 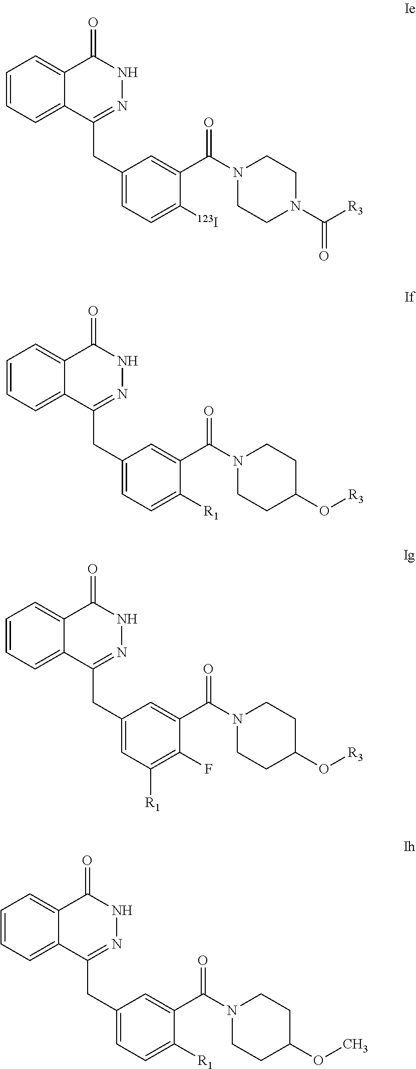 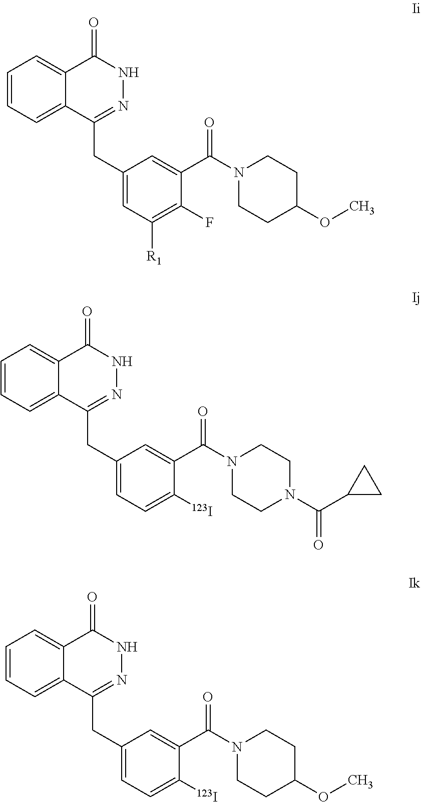
wherein, as appropriate:
R₁ is as defined in any one of claim 1 or 4 to 6;
R₃ is as defined in any one of the preceding claims; and
R₅ is as defined in any one of the preceding claims.

19. A compound according to claim 1, or a pharmaceutically acceptable salt or solvate thereof, wherein the compound is a compound of formula:

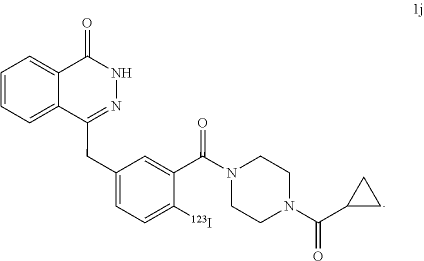 [SMILES: O=C(c1cc(Cc2n[nH]c(=O)c3ccccc23)ccc1[123I])N1CCN(C(=O)C2CC2)CC1]

20. A pharmaceutical composition comprising a compound according to any one of the preceding claims, or a pharmaceutically acceptable salt or solvate thereof, and a pharmaceutically acceptable excipient.

21. A compound according to any one of claims 1 to 19, or a pharmaceutically acceptable salt or solvate thereof, or a pharmaceutical composition according to claim 20, for use in therapy.

22. A compound according to any one of claims 1 to 19, or a pharmaceutically acceptable salt or solvate thereof, or a pharmaceutical composition according to claim 20, for use in the treatment of cancer.

23. A compound, or a pharmaceutically acceptable salt or solvate thereof, or a pharmaceutical composition, for use in the treatment of cancer according to claim 20, wherein the cancer is a cancer that expresses PARP.

24. A compound, or a pharmaceutically acceptable salt or solvate thereof, or a pharmaceutical composition, for use in the treatment of cancer according to claim 22 or claim 23, wherein the cancer is selected from glioma, glioblastoma, thyroid cancer, lung cancer (e.g. NSCLC), oesophageal cancer, head and neck cancer, tongue cancer, stomach cancer, liver cancer (e.g. HCC), neuroendocrine cancer, pancreatic cancer (e.g. PDAC), colon cancer, renal cancer, prostate cancer, breast cancers (e.g. TNBC, ductal and invasive subtypes), endometrial cancer, cervical cancer, ovarian cancer, melanoma/skin cancer and lymphoma.

25. A method of treating cancer, the method comprising administering a therapeutically effective amount of a compound according to any one of claims 1 to 19, or a pharmaceutically acceptable salt or solvate thereof, or administering a pharmaceutical composition according to claim 20.
